(19) United States
(12) Patent Application Publication
(10) Pub. No.: US 2025/0281410 A1
(43) Pub. Date: Sep. 11, 2025

(54) HIGH-THROUGHPUT PREPARATION OF MICROPARTICLES WITH PULSATILE RELEASE

(71) Applicant: William Marsh Rice University, Houston, TX (US)

(72) Inventors: Kevin McHUGH, Houston, TX (US); Tyler GRAF, Houston, TX (US)

(73) Assignee: William Marsh Rice University, Houston, TX (US)

(21) Appl. No.: 18/571,920

(22) PCT Filed: Jun. 21, 2022
(86) PCT No.: PCT/US2022/073058

§ 371 Date: Dec. 19, 2023

Related U.S. Application Data
(60) Provisional application No. 63/213,082, filed on Jun. 21, 2021.

(51) Int. Cl. A61K 9/16

(52) U.S. Cl. CPC A61K 9/1647; A61K 9/1694

(57) ABSTRACT

Disclosed herein are closed polymeric microparticles comprising an outer shell and an inner volume, wherein the outer shell has a bottom and a domed top end, and comprises at least one polymer, further wherein the inner volume comprises at least one guest agent, wherein the guest agent is a therapeutic agent, a prophylactic agent, a nutraceutical agent, or a diagnostic agent. The present disclosure also provides methods for the production of as well as methods for treatment using said microparticles.

### REFERENCE TO RELATED APPLICATIONS

The present application is a national phase application under 35 U.S.C. § 371 of International Application No. PCT/US2022/073058, filed Jun. 21, 2022, which claims the priority benefit of U.S. provisional application No. 63/213,082 filed Jun. 21, 2021, the entire contents of each of which are incorporated herein by reference.

### BACKGROUND

### I. Field

The present disclosure relates generally to the fields of materials science, chemistry, and medicine. More particularly, it concerns microparticles, methods for their preparation, and methods of their use for the treatment, diagnosis, or prevention of diseases and disorders.

### II. Description of Related Art

Previous methods for the synthesis of microparticles having pulsatile release exhibit a number of problems, including being greatly limited in the minimum size of particles that can be obtained, low production throughput, and inconsistency in production. In some methods, hollow particles composed of a thermoplastic polymer are formed through the microscopic alignment and sintering of a cup-like “base” and a polymer slab “cap” (McHugh et al., 2017; Lu et al., 2020; WO 2015/095230; WO 2019/055613). These methods require the production of two components, careful manual inspection, and are generally limited to the production of just 100-500 particles at a time. As such, there is a need for production methods require far less time and are more readily compatible with continuous processing methods (e.g., conveyer belt). Additionally, the flexibility of the molds prevents the sealing of very small particles, as does the force placed on very small particles and those composed of materials with low mechanical properties, which may induce particle fracture.

Therefore, there remains a need to develop new methods of preparing particles that address these concerns.

### SUMMARY

The present disclosure provides microparticles, methods for their manufacture, and methods for their use.

In one aspect, the present disclosure provides microparticles comprising an outer shell and an inner volume, wherein the outer shell has a bottom and a domed top end, further wherein the outer shell comprises at least one polymer, further wherein the inner volume comprises at least one guest agent, wherein the guest agent is a therapeutic agent, a prophylactic agent, a nutraceutical agent, a diagnostic agent, an excipient, or an adjuvant. In some embodiments, the microparticle has a longest dimension smaller than about 1000 μm. In further embodiments, the microparticle has a longest dimension smaller than about 650 μm. In still further embodiments, the microparticle has a longest dimension from about 50 μm to about 650 μm. In yet further embodiments, the microparticle has a longest dimension from about 400 μm to about 600 μm. In some embodiments, the bottom is flat. In other embodiments, the bottom is convex. In other embodiments, the bottom is concave. In some embodiments, the bottom is circular. In other embodiments, the bottom is polygonal. In some embodiments, the at least one polymer is a biocompatible polymer, a phase-change polymer, or a thermoplastic polymer. In some embodiments, the at least one polymer is an enteric polymer. In some embodiments, the at least one polymer is a biodegradable polymer. In some embodiments, the at least one polymer is a surface-eroding polymer. In some embodiments, the at least one polymer is a copolymer. In some embodiments, the at least one polymer is poly(lactic-co-glycolic acid) (PLGA), polylactic acid, polycaprolactone, poly(methacrylic acid), a poly(methacrylate), poly(glycerol sebacate methacrylate), or poly(glycerol sebacate acrylate). In some embodiments, the at least one polymer is two or more materials blended together, such as two different materials, such as a biodegradable polymer and plasticizer, or two of the same polymer, such as two PLGA polymers, with different properties.

In some embodiments, the at least one polymer is PLGA. the PLGA comprises lactic acid and glycolic acid monomers in a ratio from about 99:1 to about 1:99. In further embodiments, the PLGA comprises lactic acid and glycolic acid monomers in a ratio from about 90:10 to about 80:20. In still further embodiments, the PLGA comprises lactic acid and glycolic acid monomers in a ratio of about 85:15. In other embodiments, the PLGA comprises lactic acid and glycolic acid monomers in a ratio from about 55:45 to about 45:55. In further embodiments, the PLGA comprises lactic acid and glycolic acid monomers in a ratio of about 50:50. In some embodiments, the at least one polymer has a molecular weight from about 10 kD to about 200 kD. In further embodiments, the at least one polymer has a molecular weight from about 10 kD to about 100 kD, such as about 12 kD, about 12.5 kD, or about 60 kD. In some embodiments, the at least one polymer has an inherent viscosity from about 0.01 dl/g to about 1.30 dl/g using the ASTM D2857 protocol. In some embodiments, the at least one polymer has an inherent viscosity from about 0.15 d/g to about 0.25 dl/g. In some embodiments, the at least one polymer has an inherent viscosity from about 0.16 dl/g to about 0.26 dl/g. In some embodiments, the at least one polymer has an inherent viscosity from about 0.55 dl/g to about 0.75 dl/g. In some embodiments, the at least one polymer has an inherent viscosity from about 0.61 dl/g to about 0.74 dl/g. In some embodiments, at least one of the end groups of the at least one polymer is a polymerization terminating group. In some embodiments, at least one of the end groups of the at least one polymer is halo, hydroxy, amino, or —C(O)R, wherein R is hydroxy or amino; or alkoxy_{(C≤12)}, alkylamino_{(C≤12)}, dialkylamino_{(C≤12)}, aryloxy_{(C≤12)}, aralkoxy_{(C≤12)}, heteroaryloxy_{(C≤12)}, heteroaralkoxy_{(C≤12)}, or a substituted version of any of these groups. In some embodiments, the at least one polymer is a blend of two or more polymers with different properties.

In some embodiments, the outer shell further comprises a plasticizer, such as polyethylene glycol (PEG) or triethyl citrate. In some embodiments, the outer shell further comprises a pH buffering agent, such as magnesium hydroxide. In some embodiments, the outer shell has a glass transition temperature of greater than about 40° C. In some embodiments, the outer shell has a glass transition temperature of from about 30° C. to about 60° C. In some embodiments, the at least one guest agent is a therapeutic agent. In some embodiments, the at least one guest agent is a prophylactic agent. In some embodiments, the at least one guest agent is a vaccine. In some embodiments, the therapeutic agent is an immunotherapeutic agent, such as a cancer immunotherapeutic agent. In some embodiments, the volume of the at least one guest agent is less than about 25 nL. In further embodiments, the volume of the at least one guest agent is less than about 12.5 nL. In some embodiments, the volume of the at least one guest agent is from about 0.01 nL to about 12.5 nL. In some embodiments, the mass of the at least one guest agent is less than about 20 μg. In some embodiments, the mass of the at least one guest agent is from about 10 ng to about 20 μg. In some embodiments, the mass of the at least one guest agent is less than about 5 μg. In some embodiments, the mass of the at least one guest agent is about 5 μg.

In another aspect, the present disclosure provides methods of preparing a microparticle of the present disclosure, the method comprising:
a) obtaining an outer shell having a bottom and an open top end comprising an inner volume, wherein the inner volume is hollow, and wherein the outer shell comprises at least one polymer;
b) incorporating at least one guest agent into the inner volume of the outer shell to form a loaded shell, wherein the at least one guest agent is a therapeutic agent, a prophylactic agent, a nutraceutical agent, a diagnostic agent, an excipient, or an adjuvant; and
c) sealing the loaded shell, such that the loaded shell closes in on itself thereby encapsulating the at least one guest agent to form the microparticle.

In some embodiments, sealing the loaded shell comprises heating the loaded shell to a first temperature. In further embodiments, the first temperature is from about 50° C. to about 200° C. In some embodiments, the first temperature is from about 60° C. to about 100° C. In some embodiments, the heating is non-contact heating. In some embodiments, the non-contact heating comprises positioning the loaded shell in proximity to a solid surface heat source such that the loaded shell does not contact the heat source. In some embodiments, the heating comprises contacting the loaded shell with a solid surface heat source. In some embodiments, the solid surface heat source is heated to a second temperature, wherein the second temperature is from about 60° C. to 200° C. In some embodiments, the heating comprises a flow of heated liquid or gas, or a laser. In some embodiments, sealing the loaded polymeric shell comprises contacting the loaded shell with a solvent or a solvent vapor. In some embodiments, the at least one guest agent is dissolved in a solvent to form a guest solution. In some embodiments, the guest solution is incorporated into the inner volume by injection using a syringe-pump. In some embodiments, the guest solution is incorporated into the inner volume by submersion into the guest solution. In some embodiments, the at least one guest agent is lyophilized into a powder and the powder is pressed into the inner volume. In some embodiments, the method further comprises crosslinking the at least one polymer of the outer shell of the microparticle after sealing. In some embodiments, crosslinking comprises exposing the microparticle to UV radiation.

In still another aspect, the present disclosure provides microparticles prepared according to the methods of preparing a microparticle of the present disclosure.

In yet another aspect, the present disclosure provides methods of treating a disease or disorder in a patient in need thereof comprising administering to the patient an effect amount of a microparticle of the present disclosure. In some embodiments, the disease or disorder is cancer.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1H show the steps for microparticle fabrication and sealing. (FIG. 1A) PLGA films are placed under vacuum, heated above the polymer melting point or glass transition temperature, and compressed into PDMS molds to create open-faced microparticles. (FIG. 1B) Stereoscope images of open-faced microparticles. (FIG. 1C) Arrays are filled utilizing a piezoelectric dispensing system to dispense small drops of a drug solution into the core. The solvent then evaporates, which leaves behind the drug in solid form. (FIG. 1D) Representative stereoscope images of microparticles filled with 3 μg of FITC-labeled 10 kD dextran. (FIG. 1E) Filled microstructures are inverted and placed over a heat source, causing the polymer to flow and seal the open face of the microparticle. (FIG. 1F) Stereoscope image of sealed microparticles. (FIG. 1G) SEM image of microparticles before and after sealing. (FIG. 1H) Stereoscope images of microparticles heated for various times. Note: The green box indicates the suitable time range for sealing microparticles. Scale bars=100 μm.

FIGS. 2A-2I show the generation of PDMS molds and intermediate master arrays. (FIG. 2A) A 3D-printed master mold of the microparticle array on a silicon substrate is (FIG. 2B) submerged in uncured PDMS, degassed under vacuum to remove bubbles and cured at 120° C. (FIG. 2C) The structure is separated generating an inverse PDMS mold. (FIG. 2D) A photocurable polymer is poured over the master PDMS mold then (FIG. 2E) drawn into the mold under vacuum and cured at 120° C. (FIG. 2F) Delamination results in creation of a NOA array similar to but more physically robust than the master mold. (FIG. 2G) The NOA mold is (FIG. 2H) submerged in uncured PDMS that is degassed and cured at 120° C. (FIG. 2I) The structure is separated generating a PDMS inverse mold used to make microparticlesa general schematic outlining the steps involved in fabrication of the master mold used in the one-component method for the preparation of microparticles having pulsatile release.

FIG. 3 shows microstructure fidelity throughout the replication process. Top and angled views of scanning electron microscopy (SEM) image of open-faced cylinders arrays throughout the mold replication and microparticle fabrication processes. Note that all images shown were from the same location on the mold showing to show the fidelity of microstructure reproduction. Scale bars=100 μm.

FIG. 4 shows images of PLGA microparticles before sealing. PLGA microparticles of different sizes can be produced, including height, diameter, wall thickness, and shape. Note that the height of the microparticles at this stage is substantially reduced as the polymer flows and covers the cavity.

FIG. 5 shows a general schematic showing the relative height of the bottom component in the one-component method for the preparation of microparticles having pulsatile release. This capless method is facilitated by making microparticles that are taller than would traditionally be used.

FIG. 6 shows the results of filling microparticles using the four methods described in FIG. 6. Note, microparticles in the “solid filling” row appear substantially more full, but this is primarily because the material is not as well-packed. Additionally, the debris around the “flood fill” method are removed at a later step and there truly is material in the core, albeit less than the other methods.

FIGS. 7A-7C show the sealing of microparticles using different combinations of time and temperature. FIG. 7A shows PLGA_{13COOH} microparticles sealed at 100° C. for 95 seconds. FIG. 7B shows PLGA_{13COOH} microparticles sealed at 80° C. for 240 seconds. FIG. 7C shows PLGA_{13COOH} microparticles sealed at 60° C. for 20 minutes.

FIGS. 8A-8D show polycaprolactone Particles Uniformly Liquified and Sealed to Encapsulate Drugs (PULSED) microparticles. Single (FIG. 8A) unsealed and sealed (FIG. 8B) microparticles and an array of (FIG. 8C) unsealed and (FIG. 8D) sealed polycaprolactone microparticles filled with 3 μg of fluorescein sodium salt. Microparticles were sealed for 2 minutes 30 seconds at 75° C. Scale bars: white=100 μm, black=600 μm

FIG. 9 shows the sealing results. Microparticles lost height and had a more spherical shape than microparticles produced using the SEAL method (bottom right image). This allows for use of a smaller needle for injection as well as better flow properties during injection.

FIGS. 10A & 10B show the morphology of the microparticles prepared by the present one-component method. FIG. 10A shows the PDMS master mold of a large array. FIG. 10B shows a side view of an array of microparticles that have been filled and sealed.

FIG. 11A-11D show photographs of filled microparticles upon preparation (day 0). FIG. 11A shows a filled microparticle with an outer shell of PLGA 50:50, 12 kD, —COOH (Evonik 502H). FIG. 11B shows a filled microparticle with an outer shell of PLGA 50:50, IV=0.65, —COOH (Lacte16013-2). FIG. 11C shows a filled microparticle with an outer shell of PLGA 50:50, 60 kD, Ester (Evonik 505). FIG. 11D shows a filled microparticle with an outer shell of PLGA 50:50, 12.5 kD, —COOH (AkinaAP041). PLGA with different properties can be sealed by altering the time and/or temperature used.

FIG. 12 shows photographs demonstrating the degradation of microparticles constructed using various PLGA sources (502H; AP041; 5003-A) filled with fluorescently labeled dextran. The filled microparticles were incubated in phosphate buffered saline at 37° C. to initiate degradation and release of the fluorescently labeled dextran. The top series shows the microparticles on day 1 and the bottom series shows the microparticles on day 2.

FIG. 13 shows top and side view scanning electron micrographs of the microparticles. During sealing the poly(lactic-co-glycolic acid) begins to flow forming a self-sealing microparticle.

FIG. 14 shows bright field microscopy images of microparticles before sealing (left), after sealing (middle), and cut in half after sealing revealing an inner pocket (right).

FIG. 15 shows bright field microscopy images of microparticles in which l0 kDa of dextran conjugated to fluorescein isothiocyanate (FITC) has been incorporated. Incorporate of FITC in the microparticle before sealing is shown on the left and after sealing is shown on the right. Prior to sealing, PLGA can be filled with a material such as a vaccine or other drug. Sealing the microparticle results in a depot of the drug in an inner pocket inside of the core-shell microparticle. This single depot of drug enables pulsatile release.

FIGS. 16A-16D show photographs demonstrating that microparticles can be made and sealed with a chemical doped into the microparticle to allow for several different functions, such as altering release kinetics, plasticizing the polymer, and stabilizing microparticle contents prior to release. FIG. 16A shows unsealed PLGA_{13COOH} microparticles doped with 20% Mg(OH)₂. FIG. 16B shows unsealed PLGA_{13COOH} microparticles doped with 20% CaCO₃. FIG. 16C shows sealed PLGA_{13COOH} microparticles doped with 20% Mg(OH)₂. FIG. 16D shows sealed PLGA_{13COOH} microparticles doped with 20% CaCO₃.

FIGS. 17A-17E shows in vitro and in vivo release of a model drug from PULSED microparticles. FIG. 17A shows the general structure of PLGA. FIG. 17B shows normalized cumulative release of FITC-labeled 10 kDa dextran from microparticles composed of four different types of PLGA incubated in PBS at 37° C. (n=11-12). FIG. 17C shows normalized cumulative release of Alexa Fluor 647-labeled 10 kD dextran from four sets of PLGA microparticles constructed from different PLGA sources injected subcutaneously into the rear flank of SKH1-Elite mice (n=9-10). FIG. 17D Representative photographs with fluorescence overlay showing Alexa Fluor 647-labeled 10 kD dextran release from four types of PLGA microparticles in mice. Radiant efficiency is shown in units of (p/sec/cm²/sr) (μW/cm3)-1. Note, all error bars show standard error of the mean. FIG. 17E shows information regarding the PLGA materials used in the assays of FIGS. 17B-17D.

FIG. 18 shows in vitro burst release characteristics of individual microparticles. The colored bars show the period over which individual microparticles exhibited pulsatile drug release, defined as ≥75% of its total cargo release. White circles indicate the day at which a majority of cargo has been released.

FIG. 19 shows in vitro release of small molecules and macromolecules from PULSED microparticles. Graph shows release of PLGA_{13CA} microparticles filled with either 3 μg of fluorescein sodium salt (Mw=376.3 Da) salt or 3 μg of 10 kD FITC-labeled dextran (n=12). Error bars indicate the standard error of the mean.

FIGS. 20A-20C show in vitro pulsatile release of the contents within microparticles prepared by a one-component system (n>=11). FIG. 20A shows the release from a microparticle constructed from PLGA formulation 502H. FIG. 20B shows the release from a microparticle constructed from PLGA formulation B6013-2. FIG. 20C shows the release from a microparticle constructed from PLGA formulation 505.

FIGS. 21A & 21B show core-shell microparticles generated using capless sealing perform equivalently to those previously generated using the two-component sealing method in vivo. FIG. 21A shows the cumulative release % from a 502H PLGA microparticle prepared by one-component capless sealing. FIG. 21B shows the cumulative release % from a 502H PLGA microparticle prepared by a two-component sealing method (McHugh et al., 2017).

FIGS. 22A-22E show a comparison of PULSED microparticles to SEAL microparticles. SEM images of the PULSED (FIG. 22A) unsealed and (FIG. 22B) sealed as well as the SEAL microparticles (FIG. 22C) unsealed and (FIG. 22D) sealed made of PLGA_{13CA}. (FIG. 22E) Pulsatile release kinetics from PLGA_{13CA} microparticles filled with 3 μg of 10 kD FITC-labeled dextran. Graph shows normalized cumulative release of microparticles incubated in PBS at 37° C. (n=12). Error bars are standard error of the mean. Scale bars=100 μm.

FIGS. 23A-23F show a scale-up of the PULSED microparticle fabrication method and the filling consistency of the one-component capless sealing method. FIG. 23A shows the filled and sealed 22×14 microparticle array. FIG. 23B shows a close up of several microparticles within the array. All microparticles were composed of PLGA_{13COOH} and filled with 1 μg of 10 kD FITC-labeled dextran prior to sealing. Also shown are SEM images of unsealed (FIG. 23C) and sealed (FIG. 23D) PULSED microparticles. FIG. 23E shows model drug recovered from a large array (22×14) of sealed microparticles compared to the contents of 1 and 308 microparticles (n=3-5), ****p<0.0001. FIG. 23F shows in vitro cumulative release of FITC-labeled 10 kD dextran from PLGA_{13COOH} microparticles harvested from different regions (top-left, bottom-left, center, top-right, and bottom-right) of the microparticle array (n=7). Error bars indicate standard deviation. Scale bars: white=400 μm, black=1 mm.

FIG. 24 shows in vitro release from microparticles composed of PLGA blends. Graph showing the normalized release of FITC-labeled 10 kD dextran from PULSED microparticles composed of blended PLGA_{13COOH} and PLGA_{87COOR} at 1:0, 12:1, 5:1, 3:5, and 0:1 molar ratios (n=6-8).

FIG. 25 shows protein stability during encapsulation. The graph plots the temperatures experienced by microparticles during sealing for PLGA_{13CA} (Black), PLGA_{42CA} (Red), PLGA_{34E} (Green), and PLGA_{87E} (Purple). Arrows indicate when microparticles were removed from the heat source.

FIGS. 26A-26C show graphs of the retention of enzymatic activity of HRP in microparticles composed of (FIG. 26A) PLGA_{13COOH}, (FIG. 26B) PLGA_{87COOR}, or (FIG. 26C) Polycaprolactone (PCL) after filling and sealing with varying amounts trehalose as a stabilizing excipient (n=6). *p<0.05, **p<0.01, ****p<0.0001. “ns” indicates no statistical significance.

FIGS. 27A-27F show the release of Avastin from microparticles. FIG. 27A is a heat map depicting the sum of the percentage of Avastin inside the microparticles and Avastin released from the microparticles. Data is normalized to amount of Avastin filled into microparticles. “X” indicates that sampling no longer occurred due to loss of Avastin activity. Avastin activity was measured using an ELISA. The excipients that were studied were bovine serum albumin (BSA) and sorbitol: monosodium glutamate (MSG): magnesium chloride (MgCl₂). FIGS. 27B-27I show plots of the release kinetics of the microparticle formulations of FIG. 27A. The release kinetics depicted in FIG. 27B are for a microparticle with 3 μg BSA as an excipient. The release kinetics depicted in FIG. 27C are for a microparticle with 2 μg BSA as an excipient. The release kinetics depicted in FIG. 27D are for a microparticle with 5 μg sorbitol:MSG:MgCl₂ as an excipient. The release kinetics depicted in FIG. 27E are for a microparticle with 3 μg sorbitol:MSG:MgCl₂ as an excipient as an excipient. The release kinetics depicted in FIG. 27F are for a microparticle with 1 μg Sorbitol:MSG:MgCl₂ as an excipient. The graphs in FIGS. 27B-27F show the amount of Avastin inside the microparticles (blue) and the amount of Avastin released from the microparticles (red) by percentage of initial amount of Avastin.

FIGS. 28A-28C show images of syringe pump adapter. The CAD file of a syringe pump adapter is shown in FIG. 28A. A stereoscope image of syringe pump adapter is shown in FIG. 28B. A stereoscope image of syringe pump adapter attached to tubing is shown in FIG. 28C.

FIGS. 29A-29B show evaluation of alternative microparticle filling methods. Microparticles were filled with varying amounts of material by either increasing the (FIG. 29A) number of cycles or (FIG. 29B) solution concentration for the fluid filling methods.

FIG. 30 shows a plot of the viscosity limitations of each fluid filling method.

FIGS. 31A-31J show the miniaturization of PULSED microparticles. An SEM image of PULSED microparticles with a diameter of 400 μm is shown in FIG. 31A. An SEM image of PULSED microparticles with a diameter of 300 μm is shown in FIG. 31B. An SEM image of PULSED microparticles with a diameter of 200 μm is shown in FIG. 31C. An SEM image of PULSED microparticles with a diameter of 100 μm is shown in FIG. 31D. Stereoscope images of PULSED microparticles with diameter 400 μm (FIG. 31E), 300 μm (FIG. 31F). 200 μm (FIG. 31G), and 100 μm (FIG. 31H) filled with 120 μg mL-1 fluorescein sodium salt solution are also shown. FIG. 31I and FIG. 31J show images of microparticles on a nickel for scale. Scale bars: black=5 mm, white=200 μm.

### DESCRIPTION OF ILLUSTRATIVE EMBODIMENTS

Disclosed herein are microparticles, methods for their manufacture, and methods for their use, including for the treatment and/or prevention of disease.

### I. Microparticles

The microparticles of the present disclosure (also referred to as “microparticles of the present disclosure” or “microparticles disclosed herein”) are shown, for example, above, in the summary of the disclosure section, and in the claims below. They may be made using the synthetic methods outlined in the Examples section. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

The present methods produce microparticles having pulsatile release, which only the two-component SEAL process has been able to achieve as an injectable (McHugh et al., 2017; Lu et al., 2020; WO 2015/095230; WO 2019/055613). In particular, these particles may be used to achieve microparticles having pulsatile release in vivo. Larger, more complex systems can achieve pulsatile release kinetics but require surgical implantation, which is more invasive and limits potential patient acceptability and applications. The two-component SEAL method requires the fabrication of both a base and a cap. The need for two separate components significantly increases the amount of time, effort, and complexity associated with the preparation of such microparticles and thus limits the throughout and scalability of such methods. Fabrication of the base and cap each uses separate molds prepared by photolithography, deep reactive ion etching, and/or buffered oxide etching. Once the molds have been prepared, the patterns are transferred into elastomeric inverse molds. Polymer is then flowed into the elastomeric inverse mold by heating above the glass transition temperature. To seal the microparticle, the cap and base must be aligned under microscopic inspection and once again heated above the glass transition temperature to fuse the two components together.

A significant limitation of the two-component SEAL method stems from misalignment between the base and cap when constructing the microparticles. This misalignment becomes perpetuated when the size of the microparticle array grows large due to the “give” in the material of the inverse mold (e.g., polydimethylsiloxane, PDMS). This issue limits how small microparticles produced by the two-component SEAL method can be fabricated as well as limits the number of microparticles that can be constructed at one time.

In the present method, microparticles are prepared using a one-component system that does not require fabrication of a separate base and cap. The present method also obviates the need for precise alignment of a base with a cap, which can be a source of delay in production time, can hinder scalability, and requires expensive equipment. The present methods can thus produce microparticles much more efficiently. Additionally, the one-component method avoids a “flash” layer that can form webbing between sealed microparticles constructed using a two-component system. Further, the one-component method may improve microparticle shape to minimize the longest dimension and improve injectability as well as may improve the stability of a loaded drug during microparticle preparation. As the one-component methods of the present disclosure do not require the alignment step, smaller microparticles can be generated in larger arrays that improve throughput. Smaller microparticles also have the advantage of being deliverable via injection through smaller gauge needles, which are preferred for pediatric applications, such as pediatric vaccination, or catheters.

Previous methods for the production of microparticles having pulsatile release utilize a multi-component system in which a base is filled with a guest agent and then a cap is welded to the top of the base to seal the microparticles (McHugh et al., 2017; Lu et al., 2020; WO 2015/095230; WO 2019/055613). Such methods require the production of multiple components as well as careful manual inspection and alignment, and are limited to batch production of a small number of microparticles at a time. In contrast, the microparticles of the present disclosure are produced in a one-component system, wherein an open-topped thermoplastic material microparticle is created, and which can be filled with the guest agent, and then subsequent heating of the filled open particle above the glass transition temperature or melting temperature of the thermoplastic material collapses the top and seals over the filled core. This method may enable high-throughput continuous production of these filled polymer microparticles, as multiple steps/components and manual alignment are unnecessary to form arrays of microparticles using this method. Particular applications for the finished microparticles include but are not limited to delivery of vaccines or cancer immunotherapeutics.

The microparticles disclosed herein comprise an outer shell having an inner volume into which at least one guest agent may be incorporated, such as a therapeutic agent, a prophylactic agent, a nutraceutical agent, or a diagnostic agent. Non-limiting examples of therapeutic or prophylactic agents include small molecule therapeutics or nucleic acids, such as an mRNA and siRNA, or amino acids, peptides and proteins. In some embodiments, the prophylactic agent is a vaccine, such as an mRNA vaccine. The microparticles comprise a bottom and a domed top end. This bottom may be concave, convex, or flat. In some embodiments, the bottom is circular or polygonal, such that the outer shell prior to sealing is roughly cylindrical or a polygonal prism. In some embodiments, the at least one guest agent is a therapeutic or prophylactic agent, such as a vaccine or a cancer immunotherapeutic. In some embodiments, the volume of the at least one guest agent is less than about 25 nL, less than about 12.5 nL, from about 0.01 nL to about 12.5 nL, or any range derivable therein. In some embodiments, the mass of the at least one guest agent is less than about 30 μg, less than about 5 μg, from about 1 ng to about 20 μg, or from about 10 ng, 20 ng, 30 ng, 40 ng, 50 ng, 100 ng, 200 ng, 300 ng, 400 ng, 500 ng, 1 μg, 2 μg, 3 μg, 4 μg, 5 μg, 10 μg, 15 μg, to about 20 μg, or any range derivable therein.

The microparticles allow for a pulsatile release of the guest agent all at once after the microparticle shell degrades at a prescribed time (on the order of days, weeks, months, and potentially years), which can be selected by tailoring the composition of the outer shell. In some embodiments, the outer shell comprises at least one polymer. In some embodiments, the at least one polymer is a biocompatible polymer, a phase-change polymer, or a thermoplastic polymer. In some embodiments, the at least one polymer is a biodegradable or enteric polymer, such as poly(lactic-co-glycolic acid) (PLGA), polylactic acid, polycaprolactone, poly(methacrylic acid), poly(methacrylate), or derivative thereof.

In some embodiments, the at least one polymer is PLGA. In some embodiments, the PLGA comprises lactic acid and glycolic acid monomers in a ratio from about 99:1 to about 1:99, from about 90:10 to about 80:20, from about 55:45 to about 45:55, or from about 99:1, 98:2, 97:3, 96:4, 95:5, 90:10, 85:15, 80:20, 70:30, 60:40, 55:45, 50:50, 45:55, 40:60, 30:70, 20:80, 15:85, 10:90, 5:95, 4:96, 3:97, 2:98, to about 1:99, or any range derivable therein. In some embodiments the at least one polymer has a molecular weight from about 10 kD to about 200 kD, from about 10 kD to about 100 kD, or from about 10 kD, 10.5 kD, 11 kD, 11.5 kD, 12 kD, 12.5 kD, 15 kD, 20 kD, 25 kD, 30 kD, 35 kD, 40 kD, 45 kD, 50 kD, 55 kD, 60 kD, 65 kD, 70 kD, 80 kD, 90 kD, 100 kD, 125 kD, 150 kD, 175 kD, to about 200 kD, or any range derivable therein.

One of skill in that art will recognize that various polymers may be useful in the microparticles of the present disclosure. These various polymers may have different inherent viscosities. In some embodiments, the at least one polymer has an inherent viscosity from about 0.01 dl/g to about 1.00 dl/g, from about 0.15 dl/g to about 0.25 dl/g, from about 0.16 dl/g to about 0.26 dl/g, from about 0.55 dl/g to about 0.75 dl/g, from about 0.61 dl/g to about 0.74 dl/g, or any range derivable therein. The outer shell may optionally further comprise a plasticizer, such as polyethylene glycol (PEG) or triethyl citrate, and/or a pH buffering agent, such as magnesium hydroxide.

All the microparticles of the present disclosure may in some embodiments be used for the prevention and treatment of one or more diseases or disorders discussed herein or otherwise. In some embodiments, one or more of the microparticles characterized or exemplified herein as an intermediate, a metabolite, and/or prodrug, may nevertheless also be useful for the prevention and treatment of one or more diseases or disorders. Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the microparticles of the present disclosure have the advantage that they may be more amenable for administration via injection than, be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, microparticles known in the prior art, whether for use in the indications stated herein or otherwise.

### II. Methods of Manufacturing Microparticles Having Pulsatile Release

The microparticles of the present disclosure may be prepared via a one-component system in which an outer shell is created comprising an inner volume that is hollow, the outer shell having higher sides than such a shell for the fabrication for a typical microparticle having pulsatile release (McHugh et al., 2017; Lu et al., 2020; WO 2015/095230; WO 2019/055613). A guest agent is incorporated into the inner volume and to form a loaded shell and the loaded shell is closed. In some embodiments, sealing the loaded shell comprises heating the shell above its glass transition temperature or melting temperature, which collapses the top of the shell and seals the microparticle.

Microfabrication techniques (e.g., photolithography, multiphoton 3D printing, stereolithography, reactive ion etching) may be used to produce a master mold, which can then be used to create an inverse mold in an elastomeric material (e.g., polydimethylsiloxane, PDMS, perfluoropolyether tetraurethane acrylate). At least one polymer, including but not limited to enteric polymers or biodegradable polymers, such as PLGA, is then heated above its glass transition temperature or melting temperature and pressed into the mold with or without vacuum. The material is then cooled to let it solidify and removed from the mold, producing an outer shell having a bottom and an open top end comprising an inner volume, wherein the inner volume is hollow, i.e., a cup-like shape. In some embodiments, the outer shell has a glass transition temperature of greater than about 30° C., from about 30° C. to about 100° C., from about 30° C. to about 60° C., or from about 30° C., 35° C., 40° C., 45° C., 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., 145° C., to about 150° C., or any range derivable therein.

At least one guest agent can then be incorporated into the inner volume to form a loaded shell. In some embodiments, the at least one guest agent is a therapeutic agent, a prophylactic agent, a nutraceutical agent, a diagnostic agent, an excipient, or an adjuvant. In some embodiments, the at least one guest agent is a drug or vaccine. Various methods can be employed to incorporate the at least one guest agent into the inner volume, non-limiting example of which include piezoelectric or acoustic dispensing, inkjet ejection, pressure-drive liquid dispensing, manual solid filling, or batch flooding. The loaded shell is then closed to afford the microparticle. In some embodiments, sealing comprises placing the loaded shell near a heat source, such as a hot plate, and the top of the shell collapses to seal the microparticle. In some embodiments, the loaded shell is held near to the heat source for from about 10 s to about 25 min, from about 20 s to about 100 s, or from about 10 s, 20 s, 30 s, 40 s, 50 s, 1 min, 2 min, 3 min, 4 min, 5 min, 10 min, 15 min, 20 min, to about 25 min, or any range derivable therein. In some embodiments, heating is non-contact heating, such as by positioning the loaded shell in proximity to a solid surface heat source such that the loaded shell does not contact the heat source. In some embodiments, the heat source is solid surface heat source. In some embodiments, the heat source is heated to a second temperature, wherein the second temperature is from about 60° C. to about 200° C., or from about 60° C., 65° C., 70° C., 75° C., 80° C., 85° C., 90° C., 95° C., 100° C., 110° C., 120° C., 130° C., 140° C., 150° C., 160° C., 170° C., 180° C., 190° C., to about 200° C., or any range derivable therein. The microparticle is then cooled to or below room temperature. This period of cooling is often short due to the microparticles low mass/specific heat. Microparticles can then be freed from the slide using direct physical force, a pressurized gas, or dissolution of a coating between the particles and substrate, and are small enough to be injected through needles.

One of skill in the art will appreciate that there are many possible variations on this technique. For instance, the specific method to produce the molds could be varied, the molds may not require an elastomer, particularly if the shape of the microparticle is amenable, e.g., wider at the bottom (top of the inverse mold) than its top (deepest part in the inverse mold). Further, if an elastomeric material is used, the elastomeric material does not need to be PDMS, and it is contemplated that a variety of different polymers may be employed in the outer shell for various applications including but not limited to biomedical use. In some embodiments, these methods may be suitable for crystalline polymers that have a melting temperature rather than a glass transition temperature, which are not compatible with the other methods. It is contemplated that water-soluble or water-insoluble drugs could be incorporated into the microparticle. It is also contemplated that microparticles of the present disclosure could be used in environmental sensing applications, in which the material used for the outer shell would change in response to a stimulus rather than passively degrading slowly via hydrolysis, enzymatic degradation, or dissolution.

### III. Methods of Using of Microparticles Having Pulsatile Release

The microparticles having pulsatile release described herein may be used in a variety of applications, such as in the delivery of a therapeutic, a prophylactic, a nutraceutical, and/or a diagnostic agent. In some embodiments, the microparticles of the present disclosure may be used to deliver drugs, such as vaccines, immunotherapeutics, or chemotherapeutics. For vaccination, multiple populations of these microparticles can be injected at once, each primed to release at a pre determined time point by passive means (degradation). This can enable the truncation of vaccination schedules (i.e., from 3 doses administered over the course of 2-4 months) or potentially enhance the efficacy of vaccines currently administered in a single dose. Without wishing to be bound by any particular theory, the microparticles of the present disclosure may boost performance of multi-use vaccines as well (e.g. adding more doses to relatively ineffective vaccines, like RTS,S/AS01 (i.e., Mosquirix), to improve seroconversion or the duration of immunity). For cancer, these microparticles can be injected intratumorally at one time by an interventional radiologist and release immunotherapeutics or chemotherapeutics consistently over a prolonged period of time, such as weeks. This can be important for treating deep tumors which cannot be accessed frequently. Furthermore, the microparticles of the present disclosure can be incorporated in pharmaceutical compositions.

One of ordinary skill in the art will recognize that the microparticles disclosed herein can be used in any application for which any currently used microparticles may be currently employed, as well as additional ones. It is specifically contemplated that microparticles described herein can be used in applications where improved physical properties of microparticles and/or improved methods of preparing such microparticles are preferred. The microparticles of the present disclosure may possess improved properties, such as size or shape, which may enhance administration, including via injection, as compared to microparticles produced by other methods.

### IV. Combination Therapy

In addition to being used as a monotherapy, the microparticles of the present disclosure may also find use in combination with one or more other therapies. Effective combination therapy may be achieved with a single composition or pharmacological formulation that includes both agents, or with two distinct compositions or formulations, administered at the same time, wherein one composition includes a microparticle of this disclosure, and the other includes the second agent(s). Alternatively, the therapy may precede or follow the other agent treatment by intervals ranging from minutes to months.

Non-limiting examples of such combination therapy include combination of one or more microparticles of the disclosure with another anti-inflammatory agent, a chemotherapeutic agent, radiation therapy, an antidepressant, an antipsychotic agent, an anticonvulsant, a mood stabilizer, an anti-infective agent, an antihypertensive agent, a cholesterol-lowering agent or other modulator of blood lipids, an agent for promoting weight loss, an antithrombotic agent, an agent for treating or preventing cardiovascular events such as myocardial infarction or stroke, an antidiabetic agent, an agent for reducing transplant rejection or graft-versus-host disease, an anti-arthritic agent, an analgesic agent, an anti-asthmatic agent or other treatment for respiratory diseases, or an agent for treatment or prevention of skin disorders.

### V. Definitions

The definitions below supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

Some of the abbreviations used herein are as follows: PLGA refers to poly(lactic-co-glycolic acid); and PDMS refers to polydimethylsiloxane.

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanyl” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “thiocarbonyl” means —C(═S)—; “sulfonyl” means —S(O)₂—; and “sulfinyl” means —S(O)—.

In the context of chemical formulas, the symbol “—” means a single bond, “═” means a double bond, and “≡” means triple bond. The symbol “----” represents an optional bond, which if present is either single or double. The symbol “ [symbol] ” represents a single bond or a double bond. Thus, the formula

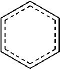

covers, for example,

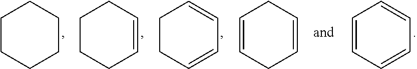

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

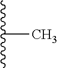

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

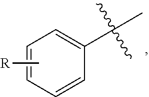

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

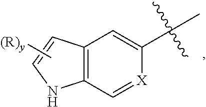

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “alkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C₁₋₄-alkyl”, “C1-4-alkyl”, “alkyl_{(C1-4)}”, and “alkyl_{(C≤4)}” are all synonymous. Except as noted below, every carbon atom is counted to determine whether the group or compound falls with the specified number of carbon atoms. For example, the group dihexylamino is an example of a dialkylamino_{(C12)} group; however, it is not an example of a dialkylamino_{(C6)} group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic 11 system. An aromatic compound or chemical group may be depicted as a single resonance structure; however, depiction of one resonance structure is taken to also refer to any other resonance structure. For example:

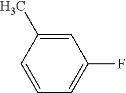

is also taken to refer to

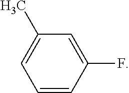

Aromatic compounds may also be depicted using a circle to represent the delocalized nature of the electrons in the fully conjugated cyclic π system, two non-limiting examples of which are shown below:

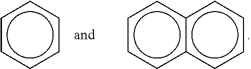

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂(i-Pr, ⁱPr or isopropyl), —CH₂CH₂CH₂CH₃ (n-Bu), —CH(CH₃)CH₂CH₃ (sec-butyl), —CH₂CH(CH₃)₂(isobutyl), —C(CH₃)₃(tert-butyl, t-butyl, t-Bu or ^{t}Bu), and —CH₂C(CH₃)₃(neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H-R, wherein R is alkyl as this term is defined above.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

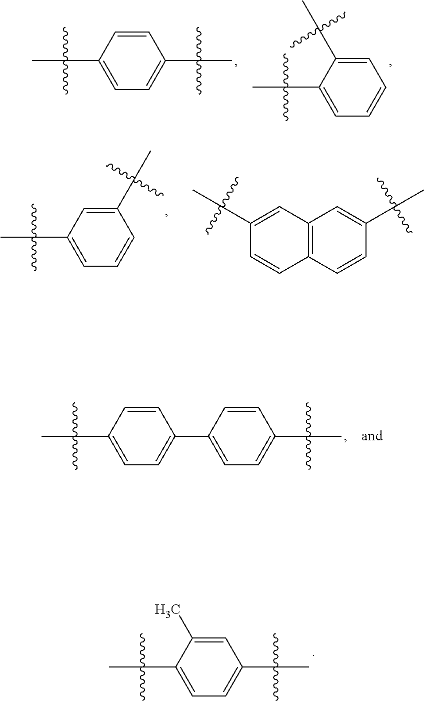

An “arene” refers to the class of compounds having the formula H-R, wherein R is aryl as that term is defined above. Benzene and toluene are non-limiting examples of arenes.

The term “aralkyl” refers to the monovalent group—alkanediyl—aryl, in which the terms alkanediyl and aryl are each used in a manner consistent with the definitions provided above. Non-limiting examples are: phenylmethyl (benzyl, Bn) and 2-phenylethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl, isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H-R, wherein R is heteroaryl. Pyridine and quinoline are non-limiting examples of heteroarenes.

The term “heteroaralkyl” refers to the monovalent group—alkanediyl—heteroaryl, in which the terms alkanediyl and heteroaryl are each used in a manner consistent with the definitions provided above. Non-limiting examples are: pyridinylmethyl and 2-quinolinyl-ethyl.

The term “alkoxy” refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂(isopropoxy), or —OC(CH₃)₃(tert-butoxy). The terms “aryloxy”, “aralkoxy”, “heteroaryloxy”, and “heteroaralkoxy” when used without the “substituted” modifier, refers to groups, defined as —OR, in which R is aryl, aralkyl, heteroaryl, and heteroaralkyl, respectively. The term “alkylthio” and “acylthio” refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The term “amido” (acylamino), when used without the “substituted” modifier, refers to the group —NHR, in which R is acyl, as that term is defined above. A non-limiting example of an amido group is —NHC(O)CH₃.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients.

An “active ingredient” (Al) or active pharmaceutical ingredient (API) (also referred to as an active compound, active substance, active agent, pharmaceutical agent, agent, biologically active molecule, or a therapeutic compound) is the ingredient in a pharmaceutical drug that is biologically active.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with microparticles means that amount or number of the microparticles which, when administered to the patient or subject, is sufficient to effect such treatment or prevention of the disease as those terms are defined below.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants, and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “seal” or “sealed” refers to an opening in a microparticle has been closed in such a manner that the microparticle no longer allows liquid or gas to escape from the inner volume.

The term “unit dose” refers to a formulation of the microparticle or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### VI. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Fabrication of Microparticles with Pulsatile Release Using Capless Sealing

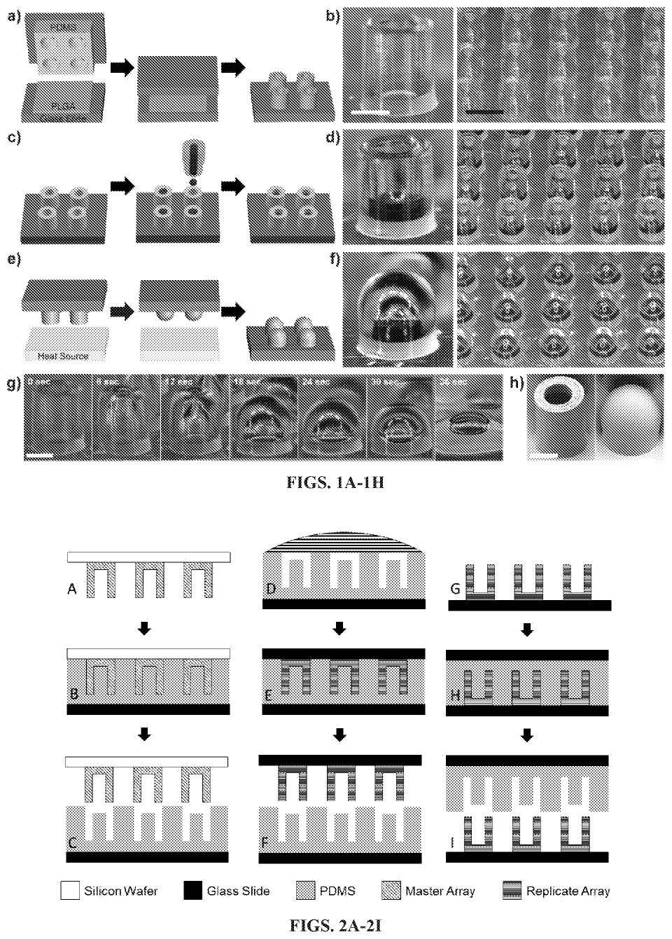

Master mold fabrication. A CAD file and Nanoscribe multiphoton 3D printer was used to print a master mold of photoresist on silicon (FIG. 1 & FIG. 2). PDMS was then cast onto that mold to create an elastomeric inverse replicate mold (FIG. 2B & FIG. 2C). To minimize potential damage to the original 3D-printed master mold, these PDMS inverse molds were used to cast a mechanically robust array using a photocurable polymer (FIGS. 2D-2F). Iteration of this step enabled the production of multiple master mold replicates, facilitating quick replication of subsequent inverse PDMS molds (FIG. 2G & FIG. 2I). All components here are re-usable, though they may be damaged after many cycles. This process facilitates minimization of damage to earlier master molds. It is contemplated that PDMS may be replaced with a fluorinated elastomer, such as perfluoropolyether tetraurethane acrylate.

Polymeric microparticles were cast by heating the polymer above its glass transition temperature and applying pressure (under vacuum) to push it into an elastomeric mold that had previously been created via soft lithograph off of a 3D-printed master mold (FIG. 1A). Four PLGA polymers were explored to vary degradation rate and release timing. All of these materials had a copolymer ratio of 50:50 lactic acid:glycolic acid, but varied in molecular weight (MW) and end group (Table 1). These materials had a MW of 13 kD and a carboxylic acid end group (PLGA_{13CA}), a MW of 42 kD and a carboxylic acid end group (PLGA_{42CA}), a MW of 34 kD and an ester end group (PLGA_{34E}), and a MW of 13 kD and an ester end group (PLGA_{87E}). PLGA films were then compressed into PDMS molds while heated above their glass transition temperature under vacuum as mentioned above to generate arrays of open-faced microparticles (FIG. 1A & FIG. 1B). PLGA_{13CA} and PLGA_{42CA} may also be known as PLGA_{13COOH} and PLGA_{42COOH}, respectively. PLGA_{34E} and PLGA_{87E} may also be known as PLGA_{34COOR} and PLGA_{87COOR}, respectively.

TABLE 1
Properties of poly(lactic-co-glycolic acid) used to fabricate microparticles.

| | | Molecular | | Lactic | |
|---|---|---|---|---|---|
| Type of | | Weight | | Acid:Glycolic | Polydispersity |
| PLGA | Material | (kDa) | End Group | Acid | Index |
| PLGA_{13CA} | Resomer ® 502H | 13 | Carboxylic | 50:50 | 2.07 |
| | | | Acid | | |
| PLGA_{42CA} | Lactel B6013-2 | 42 | Carboxylic | 50:50 | 2.70 |
| | | | Acid | | |
| PLGA_{34E} | Lactel B6010-2 | 34 | Ester | 50:50 | 1.95 |
| PLGA_{87E} | Resomer ® 505 | 87 | Ester | 50:50 | 1.95 |

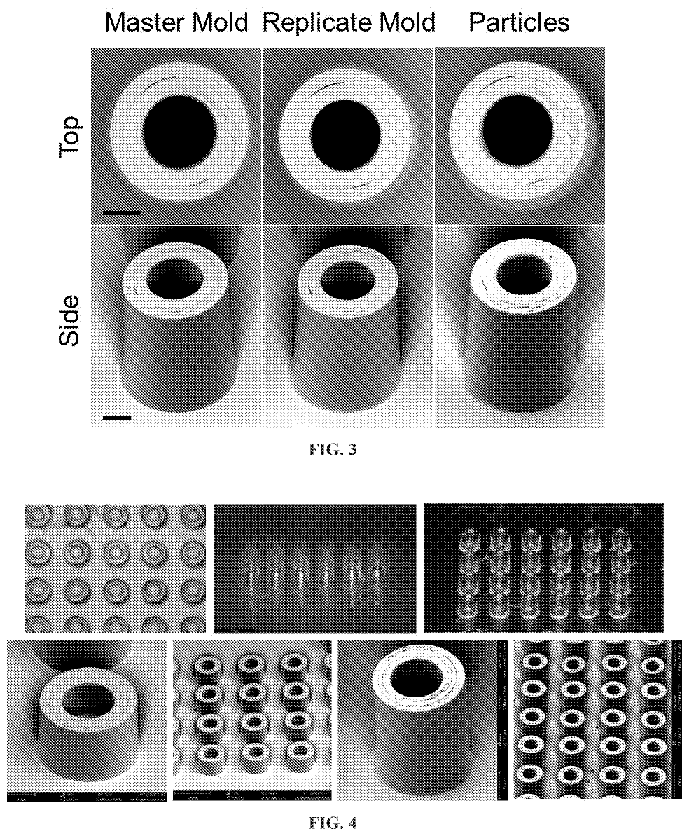

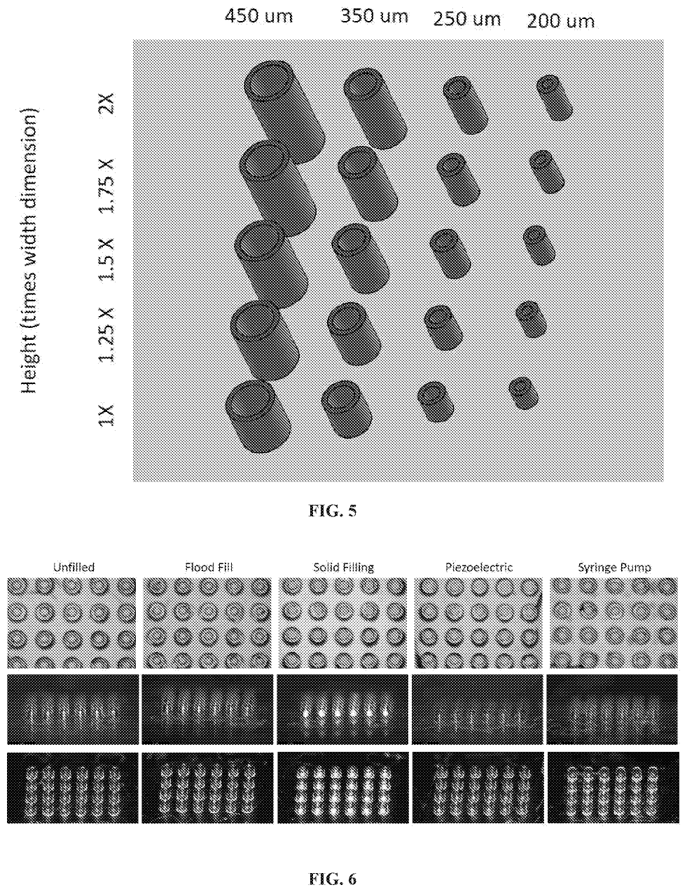

Despite the number of replications steps, the final microparticle structures remained highly consistent, reproducing even small surface topography through the process and decreasing the diameter, wall thickness, and height by only 1.8±0.3%, 3.2±1.5%, and 2.7±0.8%, respectively (FIG. 3). Poly(lactic-co-glycolic acid) (PLGA) microparticles of different sizes can be produced, including height, diameter, wall thickness, and shape (FIG. 4 and FIG. 5). The one-component capless sealing method was facilitated by making the bottom of the microparticle that are taller than would be used in the SEAL method (FIG. 5). The height of the microparticles at this stage was substantially reduced as the polymer flows and covers the cavity.

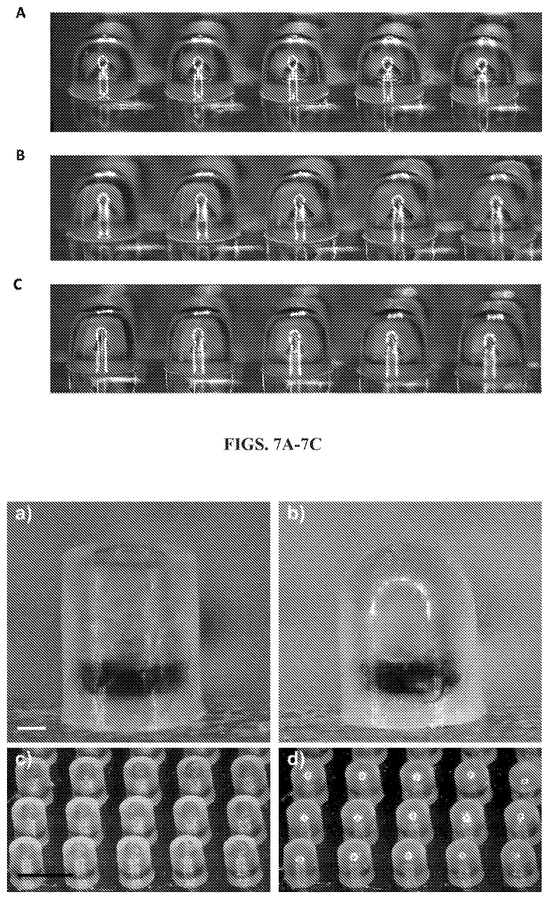

Prior to sealing, the bottom can be filled with a material such as a vaccine or other drug. Sealing the microparticle results in a depot of the drug in an inner pocket inside of the core-shell microparticle. This single depot of drug enables pulsatile release. Many different methods for filling the microparticles may be used. Here, four different methods were used to fill microparticles with a model drug: 1) piezoelectric dispensing of a drug solution (same method used in the two-component SEAL method); 2) filling will a solid lyophilized powder, which is particularly useful for incorporating hydrophobic drugs; 3) dispensing with a syringe pump attached to a custom 3D-printed tip in contact with the microparticle; and 4) flood filling where the microparticles are submerged in a drug solution of interest and centrifuged. Representations of microparticles filled by each of these four filling methods are shown in FIG. 6. All of these methods showed high levels of average filling consistency, with piezoelectric (picoliter) dispensing and syringe filling ranking the best. The filling of a microparticle via piezoelectric dispensing is also shown in FIG. 1C and FIG. 1D. It is contemplated that inkjet ejection, pressure-drive liquid dispensing, or solid (non-lyophilized) filling could also be used to fill microparticles.

The microparticles were then sealed by inverting the slide containing filled microparticles and exposing it to a heat source (FIGS. 1E-1G). This causes the polymer to flow due to a combination of surface tension and, perhaps, gravity, collapsing the top of the walls of the microparticle inward, thereby covering or enveloping the top of the cavity (FIG. 1E). Microparticles were then removed from heat and cooled to prevent undesirable deformation. To visualize and optimize this process, microparticles composed of PLGA_{13CA} were placed 1 mm over a hot plate with a surface temperature of 200° C. for 0, 6, 12, 18, 24, 30, 36 seconds and the resulting microstructures were imaged using a stereomicroscope. As seen in FIG. 1H; the open face of the cylindrical microparticles slowly began to close before fully sealing between 12 and 18 seconds exposure to the heat source. The desired structure was maintained through at least 30 seconds exposed to the heat source but was lost by 36 seconds exposure to the heat source. Therefore, a sealing time of 18 seconds was used in all subsequent experiments. A similar process was used to determine the appropriate sealing time for the other three PLGA polymers, which identified 38, 42, and 60 seconds as the best for PLGA_{42CA}, PLGA_{34E}, and PLGA_{87E}, respectively. In addition, different sealing temperatures were tested for PLGA_{13CA} to determine the corresponding required sealing time (FIGS. 7A-7C). Therefore, it can be seen that the experimental conditions and composition of the polymer may result in various preferred durations of exposure to the heat source and as such are contemplated in the present disclosure. To demonstrate that the success of the PULSED encapsulation process is not unique to PLGA or amorphous polymers, open-faced cylinders made of polycaprolactone (PCL), a biodegradable crystalline polymer were produced and sealed (FIG. 8). Microparticles may therefore be sealed using many different time and temperature combinations with many different polymer materials.

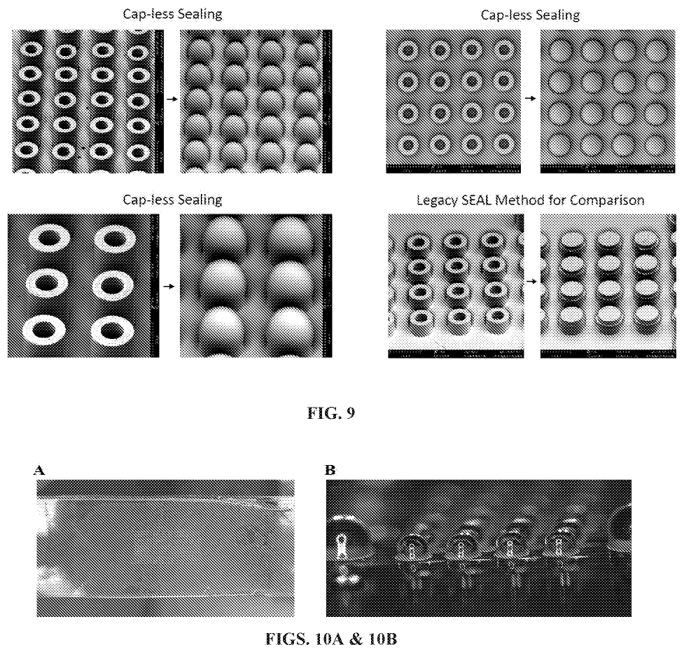

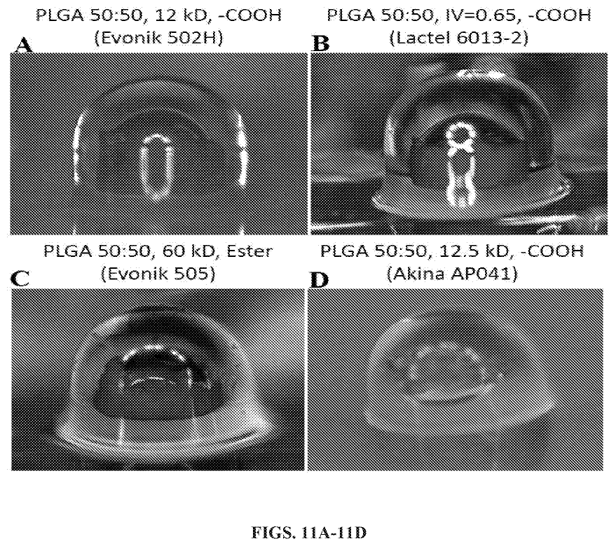

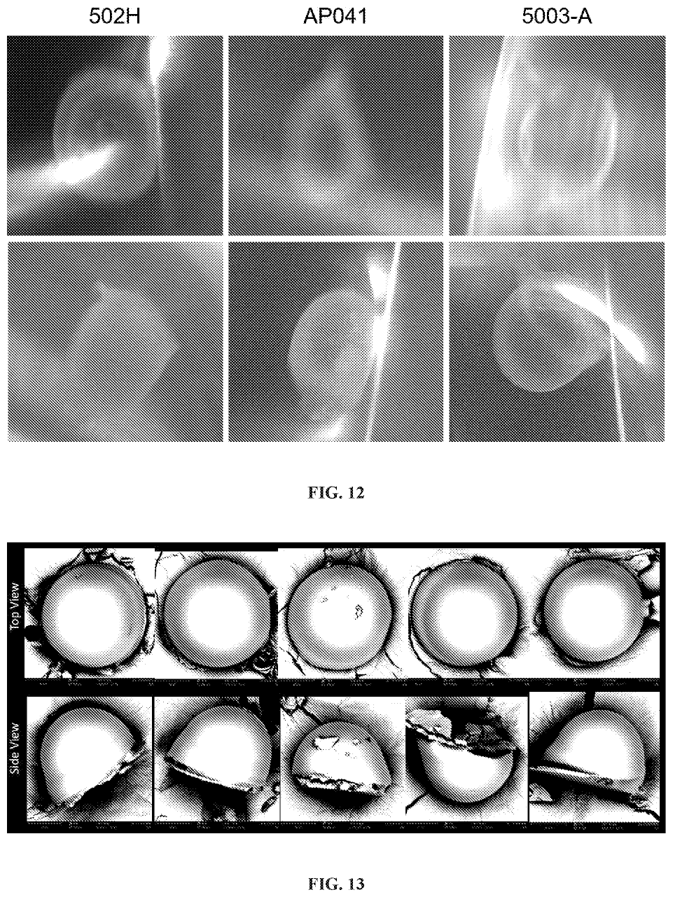

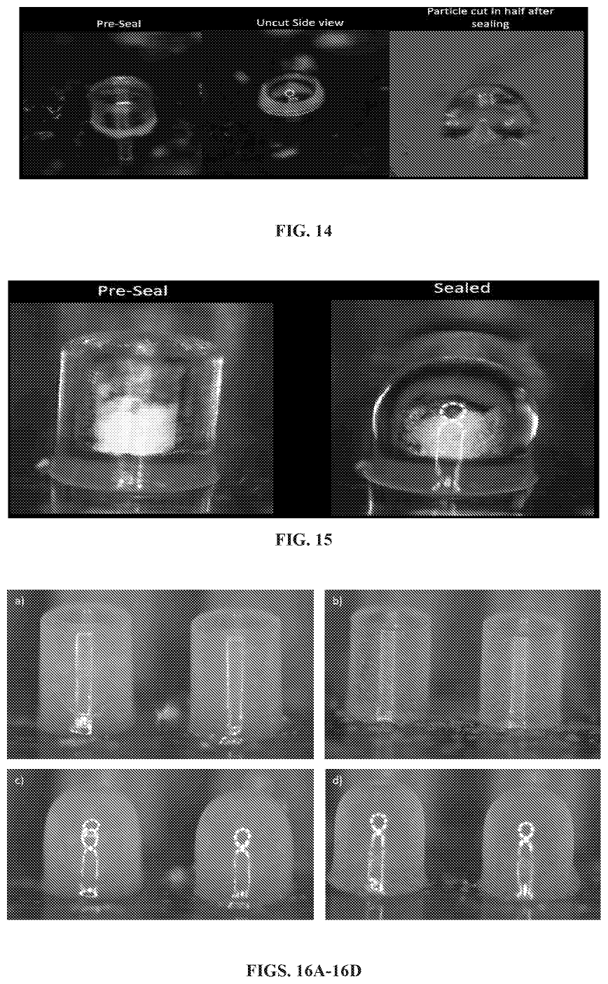

The process for forming the sealed microparticles is further illustrated in FIGS. 9-15. A glass slide of cylindrical poly(lactic-co-glycolic acid) (PLGA) microparticles was first attached to a holder. The holder was placed on top of spacers, maintaining a specific distance between the microparticles and a hot plate at a temperature above the polymer's glass transition temperature, and the microparticles were left for a specific amount of time. An image of the microparticles inverted over the heat source is shown in the upper left corner. Results of the cap-less sealing are shown in FIG. 9 along with a comparison to the two-component SEAL method. Empty microparticles and microparticles were filled by each of the filling methods described above. The elastomeric inverse mold of large array is shown in FIG. 10A, and side view of microparticles filled with fluorescently labeled dextran is shown in FIG. 10B. The one-component method for sealing the microparticles sealed lost height and had a more spherical shape than microparticles produced using the SEAL method. This allows for use of a smaller needle for injection as well as better flow properties during injection, as described below. Microparticles were able to be constructed using various sources of PLGA (502H; 6013-2; 505; AP041; 5003-A) and fluorescently labeled dextran was incorporated into the inner cavity of the bottom of the microparticle prior to sealing (FIGS. 11 & 12). Scanning electron micrographs showing flow of outer shell during sealing is shown in FIG. 13. Cutting these microparticles in half reveals an inner pocket (FIG. 14). PLGA microparticles filled with 10 kD dextran conjugated to fluorescein isothiocyanate (FITC) before and after sealing are shown in FIG. 15.

The polymer of the outer shell may be doped with a chemical to allow for several different functions. Such doped chemicals may alter the release kinetics of the payload, may plasticize the polymer, or may stabilize microparticle contents prior to release. FIG. 16 provides an example of unsealed and sealed microparticles wherein the polymer material has been doped with either Mg(OH)₂ or CaCO₃. In addition, a variety of polymers besides PLGA polymers are contemplated for use in the methods of the present disclosure. The contemplated polymers include those with melting points, such as polycaprolactone (PCL). FIG. 8 shows PCL microparticles formed using methods of the present disclosure. It is extremely difficult to form pulsatile-release microparticles from materials that do not have a glass transition temperature, such as PCL. The ability to seal polymers with a melting point broadens the polymers that are able to be used to form microparticles. A non-limiting list of materials that may be used to form the microparticles described herein via methods described herein includes Eudragit polymers, Poly(N-isopropylacrylamide), and poly [bis(p-carboxyphenoxy)]propane.

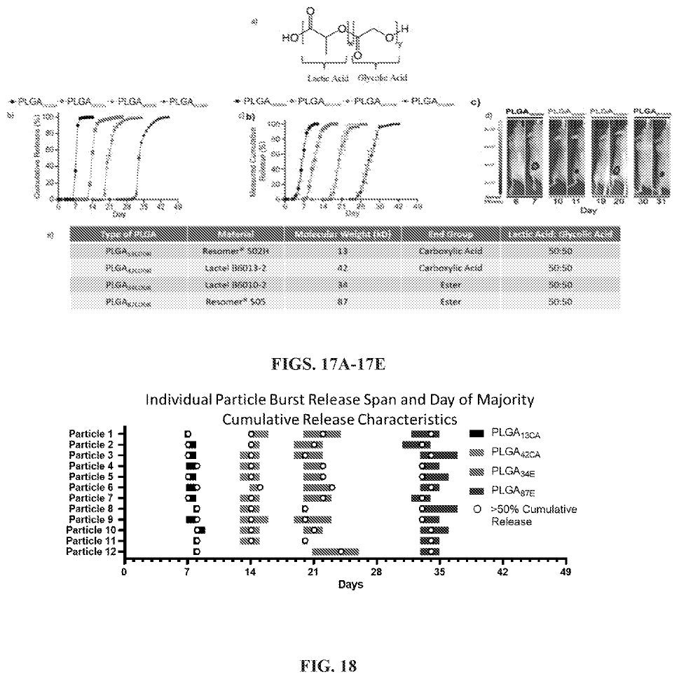

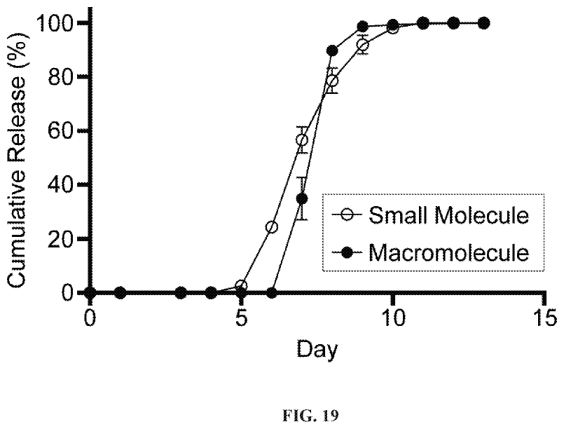

PULSED microparticles exhibit pulsatile release kinetics of their payload. The release is characterized an initial period in which no appreciable release is observed, followed by rapid release of the material they are filled with. To show that microparticles produced using the PULSED method exhibit pulsatile release, microparticles made with each PLGA were filled with fluorescein isothiocyanate-labeled 10 kD dextran as a model macromolecule drug. Release kinetics were determined by measuring release of the fluorescent molecule from microparticles incubated under agitation at 37° C. in phosphate-buffered saline (PBS) to simulate in vivo conditions. All PLGA microparticles showed pulsatile drug release after a delay that was dependent on the rate of polymer degradation. The length of delay before release was dependent on the molecular weight and end-group as PLGA_{13CA}, PLGA_{42CA}, PLGA_{34E}, and PLGA_{87E} microparticles released a majority of their cargo by 8±1, 14±0, 21±1, and 34±1 days, respectively (FIG. 17B). Increasing molecular weight and adding an ester cap both produced longer delays, which is in agreement with previously published observations (McHugh et al.) In addition, each microparticle's release was very sharp, with 75% of the model drug releasing over a period of 2±0 (PLGA_{13CA}), 3±1 (PLGA_{42CA}), 4±2 (PLGA_{34E}), and 4±1 (PLGA_{87E}) days. Further, the individual microparticles had highly similar release characteristics highlighting the consistency of the PULSED fabrication process (FIG. 18). To again show the flexibility of this pulsatile release system, the release of fluorescein sodium salt, a small molecule, from PLGA_{13CA} PULSED microparticles was evaluated. Release kinetics were similar to macromolecule both releasing on 8±1 day, though the span required to release 75% of material was slightly wider at 3±1 day (FIG. 19). Examples of small molecules and macromolecules that may be used with the microparticles described herein include but are not limited to monoclonal antibodies such as bevacizumab, anti-cancer small molecule immunotherapies such as imiquimod or resiquimod, or small molecule contrast agents such as iohexol. Other small molecules or macromolecules contemplated for use with the microparticles and methods of the present disclosure include vaccines, antibody therapeutics, peptide therapeutics, RNA therapeutics, and other small molecules therapeutics.

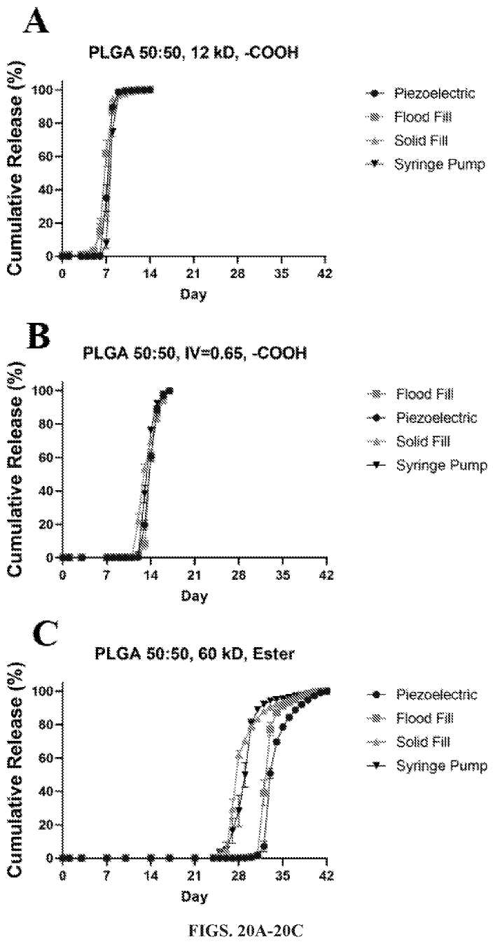

In vitro study of the cumulative release kinetics of the microparticles shows that highly pulsatile release profiles compared to other drug delivery strategies (FIGS. 20A-20C). The present method results in pulsatile release, which only the two-component SEAL process has been able to achieve as an injectable. Larger, more complex systems may be able to achieve these kinetics but require surgical implantation, which is more invasive and limits their potential patient acceptability and applications. PLGA end group, copolymer ratio, and molecular weight affect the rate of degradation and thus timing of pulsatile release. From a processing standpoint, it is preferable to alter end group and copolymer ratio before altering molecular weight to avoid an increase in glass transition temperature that requires exposure of the API to higher temperatures or to the same temperature for a longer duration of time. The properties of the PLGA can be tuned to achieve release at different time points. For example, PLGA end group, copolymer ratio, and molecular weight affect the rate of degradation and thus timing of pulsatile release. From a processing standpoint, in some instances it may be preferable to alter the end group and/or copolymer ratio before altering molecular weight to avoid an increase in glass transition temperature that requires exposure of the guest agent to higher temperatures or to the same temperature for a longer duration of time.

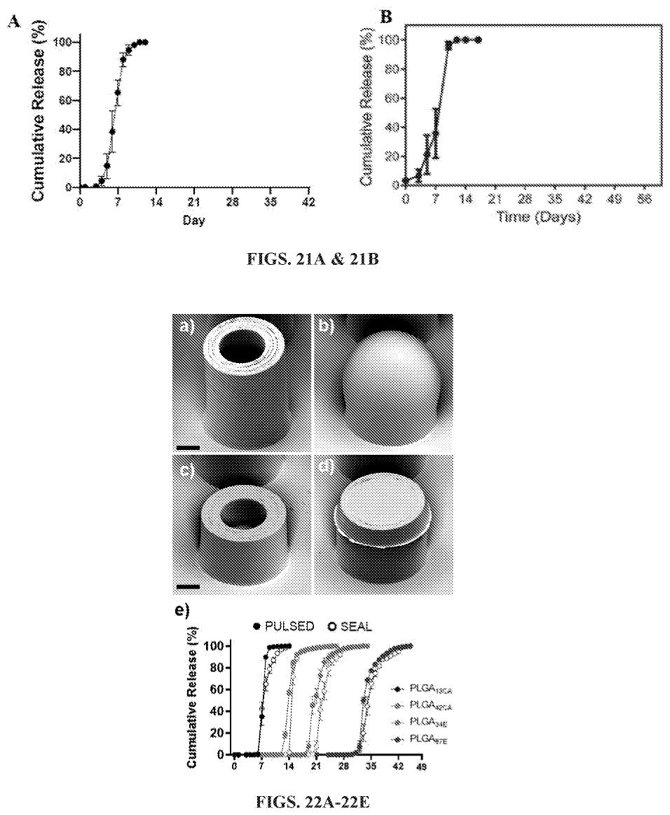

Pulsatile release microparticles in vivo. Core-shell microparticles generated using the capless sealing perform similarly to those previously generated using a two-component sealing method in vivo (FIG. 21). In vivo release is largely the same as in vitro release (compare FIG. 17B and FIG. 17C), showing pulsatile release of a macromolecule (fluorescently-labeled 10 kD dextran) after a delay that is based on PLGA properties. In one in vivo experiment, SKH1-Elite Mice were subcutaneously injected with a core-shell microparticle created from one of four PLGA materials. Core-shell microparticles were filled with Alexa Fluor 647 and release was monitored using an in vivo imaging system (IVIS). Images were collected daily around time of release. The average when each microparticle released >50% of its contents was used to quantify day of release (FIG. 17C). An n=9 to 10 was used per PLGA formulation.

To determine in vivo release kinetics, SKH1-Elite mice were subcutaneously injected with microparticles filled with Alexa Fluor 647-labeled 10 kD dextran and sealed using the PULSED method. Release of the fluorescent macromolecule was measured non-invasively using an in vivo imaging system (IVIS). Release remained pulsatile occurring on days 7±1, 11±1, 20±2, and 31±2 for PLGA_{13CA}, PLGA_{42CA}, PLGA_{34E}, and PLGA_{87E}, respectively (FIG. 17C). The fluorescent dye self-quenches when it is highly concentrated in the core, but then increases several orders of magnitude as it is released and spreads, reducing the quenching effect (McHugh et al.). Representative images of mice showing the rapid increase in fluorescence when imaged on consecutive days at the time of pulsatile release (FIG. 17D). Individual microparticle release kinetics remained highly pulsatile in vivo, releasing 75% of the total fluorescent signal over the span of 2±1 (PLGA_{13CA}), 3±1 (PLGA_{42CA}), 3±2 (PLGA_{34E}), 2±1 (PLGA_{87E}) days.

Microparticles produced using the PULSED fabrication method were compared to those created using the SEAL fabrication process—the existing “best-in-class” method for injectable pulsatile release. Images of the different morphologies produced by the sealing methods can be seen in (FIGS. 22A-22D). Drug release from microparticles produced using each method appeared very similar (FIG. 22E).

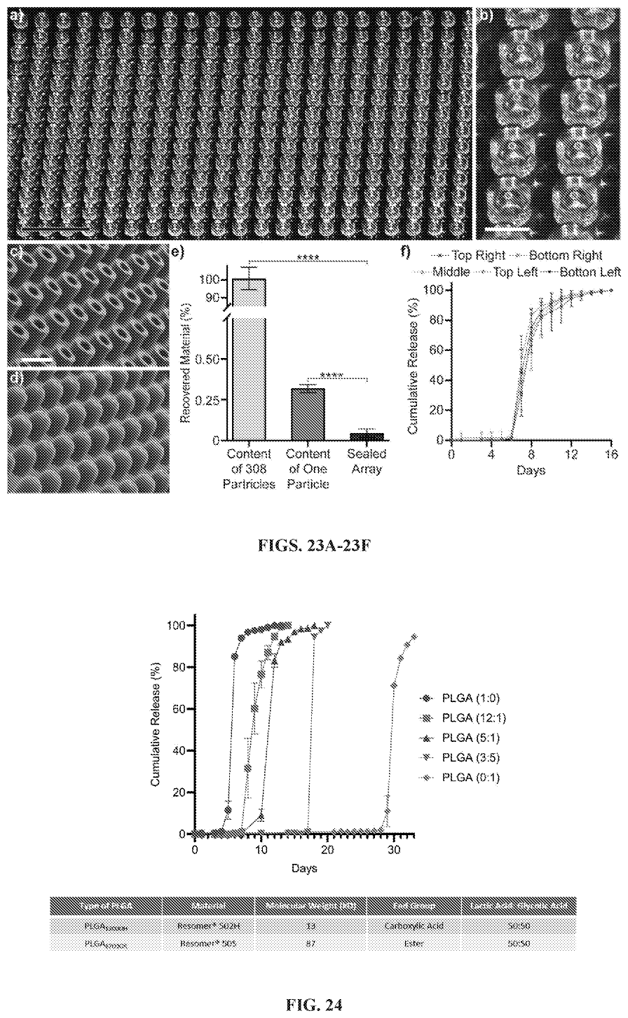

To demonstrate the scalability of the PULSED method, a 22×14 array of open-faced cylinders was generated, filled, and sealed (FIG. 23A-23C). Sealing of the 308 microparticles was highly consistent as less than one particle equivalent of material was recovered form particles after sealing, the material recovered is likely due to aberrant drops that missed particle cores during filling (FIG. 23D). In order to validate uniformity microparticle behavior, in vitro release studies were performed using microparticles harvested from five distinct regions of the array. These studies showed excellent consistency in pulsatile release, with over 50% of total material releasing by day 8±0, 8±1, 8±0, 8±1, 8±1 for the top-left, bottom-left, center, top-right, and bottom-right regions of the array respectively (p>0.05) (FIG. 23E). The large arrays of microparticles achieved 100% sealing rates (FIG. 23E), and microparticles across the array demonstrate identical release kinetics (FIG. 23F).

The methods disclosed herein may be customized to alter release kinetics, for example by forming microparticles with different microparticle compositions to achieve different delays prior to release. Blending PLGA polymers with different properties may result in payload release at intermediate, customizable time points. PLGA_{13CA} and PGLA_{87E}, which individually release on days 6±0 and 30±0 when prepared using an organic solvent, were blended at molar ratios of 12:1, 5:1 3:5 (PLGA_{13CA}:PGLA_{87E}). Microparticles produced with these molar blend ratios exhibited release at days 9±1, 12±0, and 18±0 respectively, allowing for the desired intermediate and variable delayed release. (FIG. 24). Sharp pulsatile release kinetics were also maintained with over 75% of the model therapeutic being released within 48 hours for all three blends. These results suggest, without being bound by theory, a simple path forward for customizing duration of the delay phase.

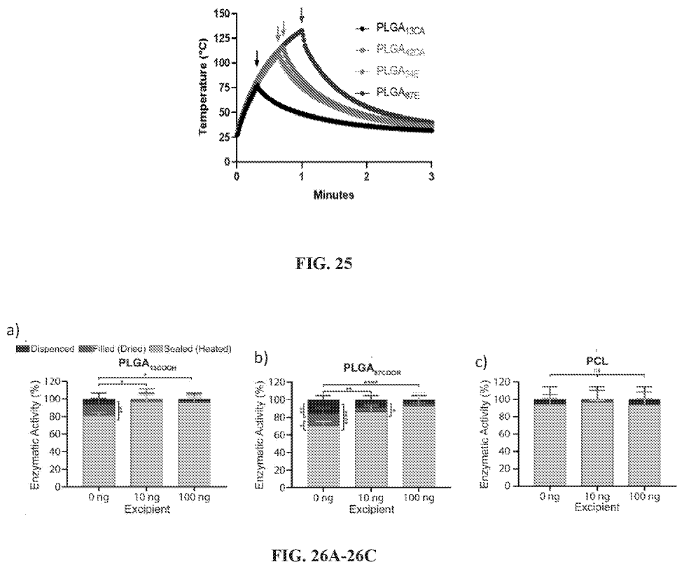

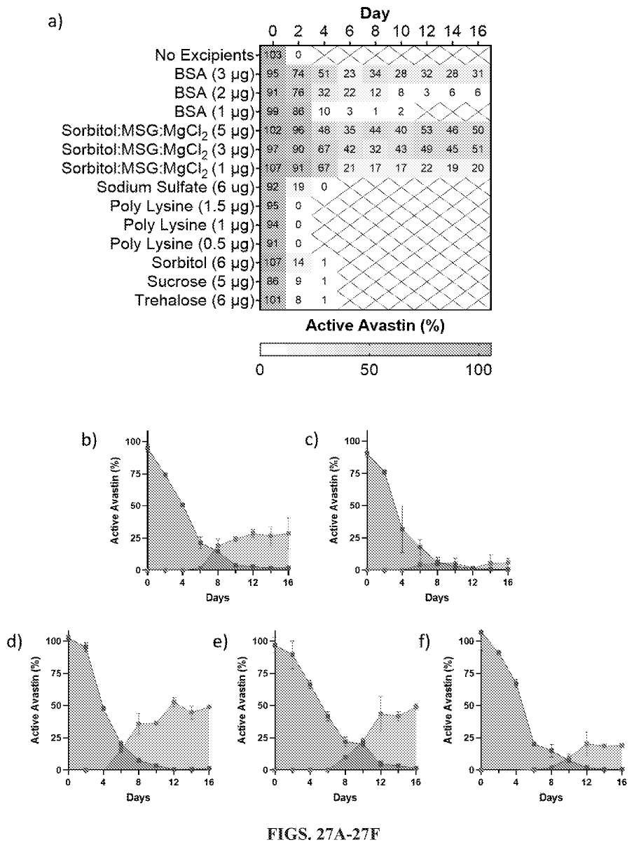

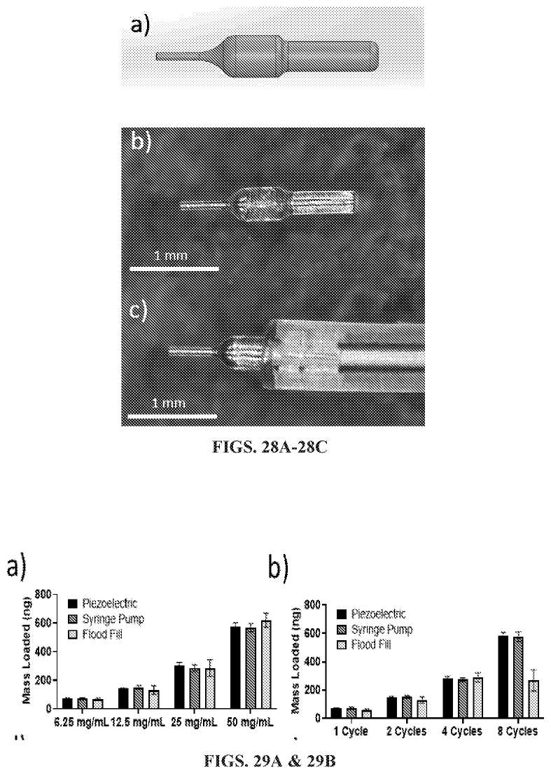

Due to concern about the thermal stress potential involved when heating up biotherapeutics which may be desirable as a payload of the microparticles described herein, the temperature of the microparticles was measured during the sealing process to determine the peak temperature and duration of exposure to elevated temperature to quantify the thermal stress that the protein might experience (FIG. 25). PLGA_{87E}, the highest molecular weight polymer studied, took the longest amount of time to close and therefore experienced the greatest amount of thermal stress, momentarily reaching a temperature of 133° C. Alternative combinations of time and temperature (e.g., more time at a lower temperature) revealed that there were options for sealing the microparticles. In general, PLGAs with higher molecular weights and ester end groups required longer sealing times and/or higher temperatures. To determine if these temperatures would damage encapsulated biotherapeutics, the stability of horseradish peroxidase (HRP), was assessed after every step in the formulation process (dispensing, drying, and sealing). These studies showed that unformulated (i.e., excipient-free) HRP maintained 81±3%% of its enzymatic activity through encapsulation in PLGA_{13CA} microparticles. However, under the harsher conditions used to seal PLGA_{87E} microparticles, only 71±5% of HRP bioactivity was retained through encapsulation. Fortunately, this loss could be mitigated by the addition of 10 ng of trehalose as a stabilizing excipient, increasing recovery to 93±15% (FIG. 26B). In order to limit protein degradation during encapsulation for long-term release applications, which might otherwise require PLGA with properties that are not amenable to low-temperature sealing, PCL microparticles filled with HRP were generated. Most PCL take years to degrade in vivo (Tsioris et al.) and, with its low melting temperature of 60° C., thermal stress was minimal, resulting in 93±7% of HRP bioactivity was retained through encapsulation, even when unformulated (FIG. 26C), providing an alternative material option for long-term release without high temperature exposure. The optimal combination of these two parameters may be payload-specific. Taken together, these results show that much of the encapsulated biological material is maintained in a bioactive state. The capability of various excipients, such as trehalose, bovine serum albumin (BSA), sorbitol, monosodium glutamate (MSG), magnesium chloride, sodium sulfate, poly lysine, and sucrose to allow for the release of active Avastin, a fragile biotherapeutic used to treat certain cancers, was examined (see FIG. 27). The addition of excipients to the polymer material during fabrication was found to result in significant amounts of Avastin remaining after release.

After demonstrating the consistency, tunability, and scalability of the PULSED encapsulation method, a microparticle filling strategy was developed to overcome the limitations of piezoelectric filling. Although robotic piezoelectric picoliter dispensers are highly programmable, precise, and accurate, these tools have several key limitations. Namely, they work in a serial manner, are incompatible with viscous solutions, and can be prohibitively expensive, limiting access to this technology and increasing cost. In an attempt to overcome these limitations, three additional filling methods were created-syringe pump filling, flood filling, and solid filling. The first method uses a standard syringe pump with a custom 3D-printed adapter that narrows the fluid flow path to a diameter that fit within the microparticle core while the flood filling method uses vacuum and/or centrifugation to pull a solution into microparticles in a batch filling process. Solid filling uses material that has been lyophilized into a compressible microfibrous structure that is then packed into microparticles. To determine if drugs could be consistently loaded into microparticles using the fluid filling methods, the filling concentration and/or the number of filling cycles we altered for each method. Altering filling concentration was found to be highly consistent with the piezoelectric, syringe pump, and flood fill method having a normalized average standard deviation of 5.41, 8.11, and 17.05% respectively. (FIG. 29A). When the concentration of drug in the filling solution cannot be easily increased due to solubility or viscosity, solution can be dispensed repeatedly into the same microparticle by allowing the previously dispensed solution to spontaneously dry and thereby providing more free volume available for the next filling cycle. This repeated approach to filling microparticles with drug also offered resulting in an average standard deviation of 5.50, and 7.03% for the piezoelectric and syringe pump respectively. The flood fill method functioned well for low cycle numbers in the flood filling method, but failed to increase loading after 4 cycles, reaching an equilibrium point at which, presumably, the amount of drug entering the microparticles in each new filling cycle was equivalent to the amount being removed (FIG. 29B). Next, since all liquid filling methods are ultimately limited by solute solubility, one additional method based on packing solid drugs into microparticle cores was pursued. To show this principle, PULSED microparticles were filled with 1 μg of imiquimod, which is minimally soluble in water (approximately 2 μg/mL). Although highly time-intensive due to the current manual process, this method demonstrated excellent utility for filling materials with extremely low solubility resulting in 0.28±0.03 μg of imiquimod loaded per particle. Using any of the liquid filling methods to fill microparticles with the same mass of imiquimod-even at its maximum concentration and full 12.57 nL microparticle core volume-would require 11,200 cycles to fill the same amount of imiquimod. Microparticles filled using each method can be seen in (FIG. 6). It was also confirmed that PULSED microparticles filled using all four methods exhibited pulsatile release kinetics (FIG. 20).

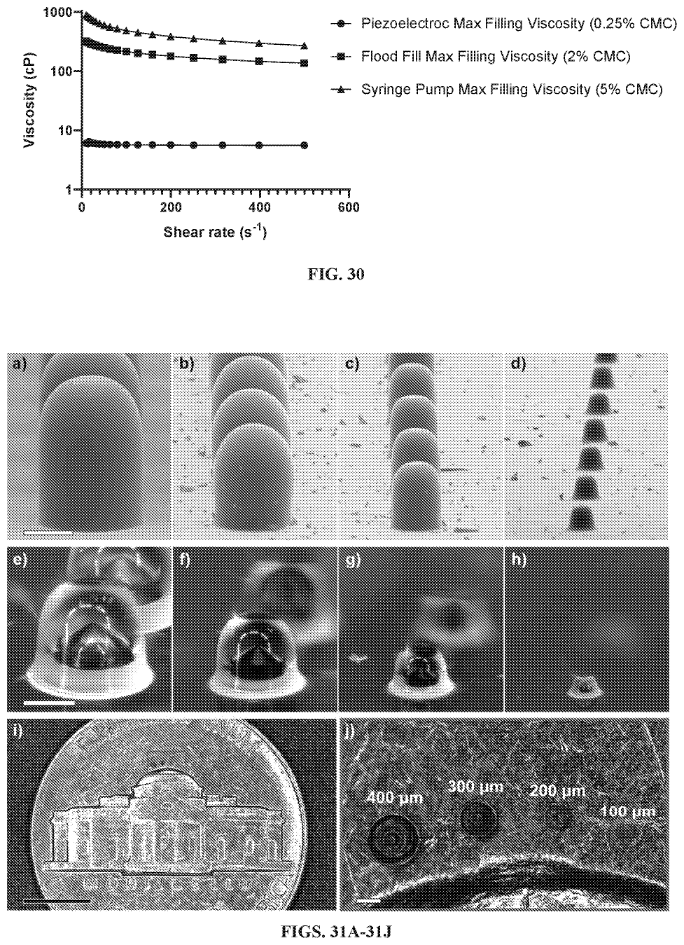

With respect to commercial scale-up, dispensing a higher concentration solution into microparticles is preferred over increasing cycle numbers because it requires far less processing time; however, a key limitation of piezoelectric dispensing is its inability to dispense viscous solutions. If a solution is visually observed to be even slightly more viscous than water, it is unlikely to be compatible with piezoelectric dispensing. To determine if the viscosity limitations of the piezoelectric device could be overcome using our syringe pump or flood filling method, we prepared carboxymethylcellulose (CMC) sodium salt solutions and determined their compatibility with filling. The piezoelectric dispensing, flood filling, and syringe pump methods were able to fill CMC solutions with maximum viscosities of 5.6, and 17.8, and 193 cP (the highest tested), respectively, at a shear rate of 200 s⁻¹ (FIG. 30). By offering compatibility with solutions that are more than 34-fold more viscous than those suitable for piezoelectric dispensing, syringe pumping is able to fill far more concentrated drug solutions, reducing microparticle fabrication cost and increasing throughput. Flood filling did not offer substantial gains in solution viscosity relative to piezoelectric dispensing, but could still be useful in a minority of use cases in which the drug is highly water-soluble and inexpensive since it enables batch filling rather than the serialized filling of individual microparticles.

Further, one major flaw of the two-component SEAL method is the misalignment between particles that becomes perpetuated when the size of the microparticle array grows large due to the “give” in the PDMS. Without the need to align microparticles, the present one-component method may allow for the fabrication of microparticles that are much smaller and therefore more easily injected. This may improve injectability and enable smaller needles, including those preferred for pediatric vaccination.

Although microparticles described above pass readily through an 18-gauge needle, which is used for some applications, such as blood collection, smaller microparticles would enable the use of narrower needles and improve patient acceptability. Therefore, microparticles were further miniaturized while maintaining the same height:diameter:wall thickness ratio of 5:4:1. Miniaturized particles were easily sealed (FIG. 30A-30D); however, due to the high aspect ratio of the core 4:2 (height:diameter) and droplet diameter, the piezoelectric dispensing tool was only able to fill microparticles with sufficient accuracy and reproducibility that has a target three-quarters the size of our primary microparticles scale having an outer diameter of 300 μm. To fill even smaller particles, different versions of the syringe pump adapter were printed to allow the tip to fit inside of the core of the smaller microparticles. Using this loading technique, microparticles were filled that were able to flow in a 30-gauge needle (FIG. 30E-30J), which is smaller than the size used for most pediatric vaccinations (22- to 25-gauge) and on par with the smallest commonly used needles for insulin administration (29- to 31-gauge).

All the microparticles, formulations, and methods disclosed and claimed herein can be made and executed without undue experimentation in light of the present disclosure. While the microparticles, formulations, and methods of this disclosure have been described in terms of preferred embodiments, it will be apparent to those of skill in the art that variations may be applied to the microparticles, formulations, and methods, as well as in the steps or in the sequence of steps of the method described herein without departing from the concept, spirit, and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references to the extent that they provide exemplary procedural or other details supplementary to those set forth herein, are specifically incorporated herein by reference.
Anderson, Practical Process Research & Development—A Guide for Organic Chemists, 2^{nd} ed., Academic Press, New York, 2012.
Handbook of Pharmaceutical Salts: Properties, and Use, Stahl and Wermuth Eds., Verlag Helvetica Chimica Acta, 2002.
Reagan-Shaw et al., FASEB J., 22(3):659-661, 2008 Smith, March's Advanced Organic Chemistry: Reactions, Mechanisms, and Structure, 7^{th} Ed., Wiley, 2013.
WO 2015/095230
WO 2019/055613
Lu et al., Sci. Transl. Med., 12:6606, 2020.
McHugh et al., Science, 357:1138-1142, 2017.
Tsioris et al., Adv. Funct. Mater 22:330, 2012

1. A microparticle comprising an outer shell and an inner volume, wherein the outer shell has a bottom and a domed top end, further wherein the outer shell comprises at least one polymer, further wherein the inner volume comprises at least one guest agent, wherein the guest agent is a therapeutic agent, a prophylactic agent, a nutraceutical agent, or a diagnostic agent.

2.-38. (canceled)

39. The microparticle of claim 1, wherein the outer shell has a glass transition temperature of greater than about 30° C.

40. The microparticle of claim 39, wherein the outer shell has a glass transition temperature of from about 30° C. to about 60° C.

41.-69. (canceled)

70. A method of preparing a microparticle of claim 1, the method comprising:
a) obtaining an outer shell having a bottom and an open top end comprising an inner volume, wherein the inner volume is hollow, and wherein the outer shell comprises at least one polymer;
b) incorporating at least one guest agent into the inner volume of the outer shell to form a loaded shell, wherein the at least one guest agent is a therapeutic agent, a prophylactic agent, a nutraceutical agent, a diagnostic agent, an excipient, or an adjuvant; and
c) sealing the loaded shell, such that the loaded shell closes in on itself thereby encapsulating the at least one guest agent to form the microparticle.

71. The method of claim 70, wherein sealing the loaded shell comprises heating the loaded shell to a first temperature.

72. The method of claim 71, wherein the first temperature is from about 50° C. to about 200° C.

73. (canceled)

74. The method of claim 71, wherein the heating is heating via radiation or irradiation.

75. The method of claim 71, wherein the heating is non-contact heating.

76. The method of claim 75, wherein the non-contact heating comprises positioning the loaded shell in proximity to a solid surface heat source such that the loaded shell does not contact the heat source.

77. The method of claim 71, wherein the heating comprises contacting the loaded shell with a solid surface heat source.

78. The method of claim 76, wherein the solid surface heat source is heated to a second temperature, wherein the second temperature is from about 60° C. to 200° C.

79. The method of claim 71, wherein the heating comprises a flow of heated liquid or gas, or a laser.

80. The method of claim 70, wherein sealing the loaded polymeric shell comprises contacting the loaded shell with a solvent or a solvent vapor.

81.-86. (canceled)

87. A method of making a microparticle comprising:
(A) forming a polymeric shell, wherein the polymer shell has a length and width and the width is less than 500 m;
(B) depositing at least one guest agent in a core formed by the polymeric shell; and
(C) collapsing an end of the shell on itself to seal the guest agent into the polymeric shell.

88. The method of claim 87, wherein the microparticle comprises an aspect ratio of the length and the width of greater than 1:1.

89. The method of claim 87, wherein the aspect ratio is greater than about 1.25:1.

90. The method of claim 88, wherein the aspect ratio is greater than about 1.5:1.

91. The method of claim 87, wherein the method further comprises altering the length and width of the polymeric shell after the at least one guest agent is deposited.

92. The method of claim 87, wherein the method further comprises altering the length and width of the polymeric shell after collapsing an end of the polymeric shell.

93. (canceled)

94. A microparticle comprising:
(A) a biodegradable polymeric shell, wherein the biodegradable polymeric shell comprises a first side and a second side, a maximum dimension of the biodegradable polymeric shell of 1 mm with an aspect ratio of greater than 1:1, and the first side and second side form an enclosed cavity with at least one of the first side or the second side being convex; and
(B) at least one guest agent retained in the enclosed cavity.

95.-99. (canceled)
